(19) United States
(12) Patent Application Publication
(10) Pub. No.: US 2023/0225606 A1
(43) Pub. Date: Jul. 20, 2023

(54) CYSTOSCOPE WITH DOCKING INTERCHANGEABLE TOOLS

(71) Applicant: Prodeon Medical, Inc., Sunnyvale, CA (US)

(72) Inventors: Al Burdulis, Sunnyvale, CA (US); Craig Purdy, Sunnyvale, CA (US); Gary Daniel Zaretzka, Sunnyvale, CA (US)

(73) Assignee: Prodeon Medical, Inc., Sunnyvale, CA (US)

(21) Appl. No.: 18/155,856

(22) Filed: Jan. 18, 2023

Related U.S. Application Data
(60) Provisional application No. 63/300,800, filed on Jan. 19, 2022.

(51) Int. Cl. A61B 1/307; A61B 1/018; A61B 1/015

(52) U.S. Cl. CPC A61B 1/307; A61B 1/015; A61B 1/018

(57) ABSTRACT

A cystoscope and system are disclosed in which a repository of the cystoscope is configured to receive a cartridge tool and provide access to a working channel of an elongated shaft of the cystoscope. The handle of the cystoscope has a first control for actuating a movable element of the repository. The movable element is configured to engage a cartridge tool received within the repository and thereby implement a functionality of the cartridge tool when the first control is actuated.

### RELATED APPLICATIONS

This application claims priority to U.S. Provisional Application No. 63/300,800, filed Jan. 19, 2022. The priority of this application is expressly claimed, and the disclosure is hereby incorporated by reference in its entirety.

### FIELD OF THE PRESENT DISCLOSURE

This disclosure relates to devices for managing or treating body tissues obstructing a hollow body lumen, such as the prostatic lobe tissues obstructing the urethra and more particularly, to devices and methods for facilitating the use of cystoscopes for diagnostic and therapeutic applications.

### BACKGROUND

The prostate is a walnut-shaped gland that wraps around the urethra through which urine is expelled from the bladder and plays a crucial role in the reproductive system of men. Although the gland starts out small, it tends to enlarge as a man ages. An excessively enlarged prostate results in a disease known as benign prostatic hyperplasia (BPH). Benign prostatic hyperplasia (BPH) refers to the abnormal, but non-malignant (non-cancerous) growth of the prostate observed very commonly in aging men. BPH is a chronic condition and is associated with the development of urinary outflow obstruction or luminal narrowing in the prostatic urethra. Bladder outlet obstruction (BOO) refers to a blockage at the base of the bladder that reduces or stops the flow of urine into the urethra and may be secondary to BPH. A range of related disorders referred to collectively as Lower Urinary Tract Symptoms (LUTS) can result, including sexual dysfunction, frequent urination, difficulty in voiding urine, urinary retention, urinary leakage, and urinary tract and bladder infections that worsen as the abnormal growth in the prostate enlarges and progresses.

Cystoscopes are an important tool for use in diagnostic or therapeutic procedures related to conditions such as those noted above, whether surgical or less invasive techniques include implanting an expandable device within the prostatic urethra that is designed to increase the diameter of the urethra. Accordingly, cystoscopes are used to examine the urinary tract and often times perform simple procedures to diagnose and correct urinary tract issues. They are composed of a handpiece with a light source and either a fiberoptic lens for direct viewing or a system that contains or connects to a camera at the end of the device. Cystoscopes may be rigid or flexible, with the flexible versions offering more patient comfort by allowing steering of the tip via a control on the handle. Cystoscopes also often include irrigation, so that delivery of saline or other suitable fluid introduced through a suitable port, such as a luer connection, is communicated through a working channel to the distal end of the device to facilitate viewing and imaging by clearing away debris/blood from the camera’s view. Procedures may be performed with the cystoscope by advancing elongated tools or instruments through the working channel. The tools can be simple graspers or baskets or more complex device delivery and/or retrieval tools but generally must fit through the irrigation port channel.

For example, FIG. 1 shows a conventional cystoscope 1 with its distal end routed through the urethra 2 and positioned in the bladder 3 of a patient. Notably, a separate tool 4 may be introduced through a port 5 of cystoscope 1. In such prior art embodiments, tool 4 typically has controls 6 that must be actuated to implement its functionality, such as deploying an expandable implant to help restore patency to the urethra such as described in co-pending, commonly-assigned U.S. Pat. No. 11,497,637 which is incorporated herein by reference in its entirety, or retrieving such an implant if warranted. This is in addition to the manipulation of cystoscope 1 as it is guided to the proper location and stabilized. As will be appreciated, such additional tools usually increase the bulk and weight of the construct and require an additional operator to manipulate the controls.

It would be beneficial to achieve the functionality provided by the separate tools while reducing the bulk and/or weight of the devices used to perform the procedure. Likewise, it would also be desirable to facilitate operation of the cystoscope and manipulation of controls used to achieve the functionality of the separate tools by a single operator. Among other benefits, such techniques would represent a simplification of the procedure and can reduce the overall time needed. The present disclosure satisfies these and other needs as detailed in the specification and drawings.

### SUMMARY

This disclosure is directed to a cystoscope having a handle, an elongated shaft that defines at least one working channel, a repository configured to receive a cartridge tool and provide access to the working channel and a first control for actuating a movable element of the repository, wherein the movable element is configured to engage a cartridge tool received within the repository and thereby implement a functionality of the cartridge tool when the first control is actuated.

In one aspect, the cystoscope also has an irrigation port in fluid communication with the working channel, wherein the irrigation port is separate from repository. The repository may have a seal configured to restrict egress of irrigation fluid from the working channel when a cartridge tool is docked within the repository.

In one aspect, the handle further may have a steering control configured to cause selective deflection of a distal end of the elongated shaft. The handle may also have a steering lock configured to maintain the distal end of the elongated shaft at a desired deflection.

In one aspect, the repository may have a recess such that at least a portion of a docked cartridge tool is within a profile of the handle.

In one aspect, the cystoscope also includes at least one cartridge tool, wherein the cartridge tool has an elongated distal portion configured to extend into the working channel and has a body that releasably attaches to the repository. The cartridge tool may have a movable element that is engaged by the movable element of the handle when the cartridge tool is docked within the repository such that manipulation of the first control implements a functionality of the cartridge tool.

This disclosure is also directed to a cystoscope system. The system may include a cystoscope having a handle, an elongated shaft that defines at least one working channel, a repository configured to receive a cartridge tool and provide access to the working channel and a first control for actuating a movable element of the repository, wherein the movable element is configured to engage a cartridge tool docked within the repository and thereby implement a functionality of the cartridge tool when the first control is actuated and at least one cartridge tool, wherein the cartridge tool has an elongated distal portion configured to extend into the working channel and has a body that releasably attaches to the repository.

In one aspect, the cartridge tool may have a movable element that is engaged by the movable element of the handle when the cartridge tool is docked within the repository such that manipulation of the first control implements a functionality of the cartridge tool.

In one aspect, the cystoscope may have an irrigation port in fluid communication with the working channel such that the repository includes a seal configured to restrict egress of irrigation fluid from the working channel when the cartridge tool is docked within the repository.

In one aspect, the cystoscope system may have a plurality of cartridge tools. At least two of the plurality of cartridge tools may have different functionalities and/or at least two of the plurality of cartridge tools may have expandable implants of different sizes.

This disclosure is also directed to a method of employing a cystoscope. The method may include providing a cystoscope having a handle, an elongated shaft that defines at least one working channel, a repository configured to receive a cartridge tool and provide access to the working channel and a first control for actuating a movable element of the repository, docking a cartridge tool with the cystoscope such that a distal elongated portion of the cartridge tool extends through the working channel and a body of the cartridge tool releasably attaches within the repository, wherein docking the cartridge tool causes the movable element of the repository to engage the cartridge and actuating the first control to implement a functionality of the cartridge tool.

In one aspect, irrigation fluid may be supplied to the working channel through an irrigation port, wherein docking the cartridge tool engages a seal that restricts egress of irrigation fluid from the working channel into the repository.

In one aspect, a steering control of the handle of the cystoscope may be operated to cause selective deflection of a distal end of the elongated shaft. Further, a steering lock of the handle of the cystoscope may be operated to maintain the distal end of the elongated shaft at a desired deflection.

In one aspect, the cartridge tool may have a movable element that is engaged by the movable element of the handle when the cartridge tool is docked within the repository such that manipulation of the first control implements a functionality of the cartridge tool.

In one aspect, the cartridge tool may be exchanged with a different cartridge tool.

### BRIEF DESCRIPTION OF THE DRAWINGS

Further features and advantages will become apparent from the following and more particular description of the preferred embodiments of the disclosure, as illustrated in the accompanying drawings, and in which like referenced characters generally refer to the same parts or elements throughout the views, and in which:

FIG. 1 is a cross-section of the male anatomy comprising the lower portion of the bladder, and the prostatic urethra in a physiological configuration typical of a patient suffering from BPH, showing an example of prior art techniques that involve routing a cystoscope and a separate tool through the prostatic urethra to access the bladder and the adjacent prostrate.

FIG. 2 schematically depicts an elevational view of a cystoscope according to an embodiment.

FIG. 3 schematically depicts a detail view of a handle of the cystoscope of FIG. 2, showing controls for steering and for implementing a functionality of a cartridge tool according to an embodiment.

FIG. 4 schematically depicts a detail view of a handle of the cystoscope of FIG. 2, showing a repository for docking a cartridge tool according to an embodiment.

FIG. 5 schematically depicts an elevational view of a cartridge tool for docking with a cystoscope according to an embodiment.

FIGS. 6 and 7 schematically depicts different views of a handle of a cystoscope according to an embodiment.

FIG. 8 schematically depicts a cut away view showing details regarding the selectively deflectable distal end of the cystoscope of FIG. 2 according to an embodiment.

FIG. 9 schematically depicts a repository of the handle of a cystoscope for docking a cartridge tool according to an embodiment.

FIGS. 10 and 11 schematically depict usage of a cartridge tool actuator of a cystoscope to implement a functionality of a cartridge tool according to an embodiment.

FIG. 12 schematically depicts usage of a cartridge tool actuator of a cystoscope to implement a functionality of a cartridge tool in an initial, undeployed position according to an embodiment.

FIG. 13 schematically depicts usage of a cartridge tool actuator of a cystoscope to implement a functionality of a cartridge tool in a fully deployed position according to an embodiment.

### DETAILED DESCRIPTION

At the outset, it is to be understood that this disclosure is not limited to particularly exemplified materials, architectures, routines, methods or structures as such may vary. Thus, although a number of such options, similar or equivalent to those described herein, can be used in the practice or embodiments of this disclosure, the preferred materials and methods are described herein.

It is also to be understood that the terminology used herein is for the purpose of describing particular embodiments of this disclosure only and is not intended to be limiting.

The detailed description set forth below in connection with the appended drawings is intended as a description of exemplary embodiments of the present disclosure and is not intended to represent the only exemplary embodiments in which the present disclosure can be practiced. The term “exemplary” used throughout this description means “serving as an example, instance, or illustration,” and should not necessarily be construed as preferred or advantageous over other exemplary embodiments. The detailed description includes specific details for the purpose of providing a thorough understanding of the exemplary embodiments of the specification. It will be apparent to those skilled in the art that the exemplary embodiments of the specification may be practiced without these specific details.

For purposes of convenience and clarity only, directional terms, such as top, bottom, left, right, up, down, over, above, below, beneath, rear, back, and front, may be used with respect to the accompanying drawings. These and similar directional terms should not be construed to limit the scope of the disclosure in any manner.

Unless defined otherwise, all technical and scientific terms used herein have the same meaning as commonly understood by one having ordinary skill in the art to which the disclosure pertains. Moreover, as used in this specification and the appended claims, the singular forms “a,” “an” and “the” include plural referents unless the content clearly dictates otherwise.

With respect to orientation of the various structures and anatomical references described herein, the term “proximal” and “distal” are relative to the perspective of the medical professional, such as a urologist, who is manipulating the cystoscope system of the disclosure. Accordingly, those features of the cystoscope system held by the hand of the urologist are at the “proximal” end and the assembled system and the imaging sensor is located at the “distal” end of the cystoscope system.

As noted above, the techniques of this disclosure involve providing a system incorporating a cystoscope and an interconnecting cartridge tool. The cystoscope includes a handle configured to facilitate guiding a distal end of the cystoscope through a patient’s anatomy and includes at least a first control that is configured to actuate a movable element of the cartridge tool. Accordingly, a single user may operate both the cystoscope and the cartridge tool.

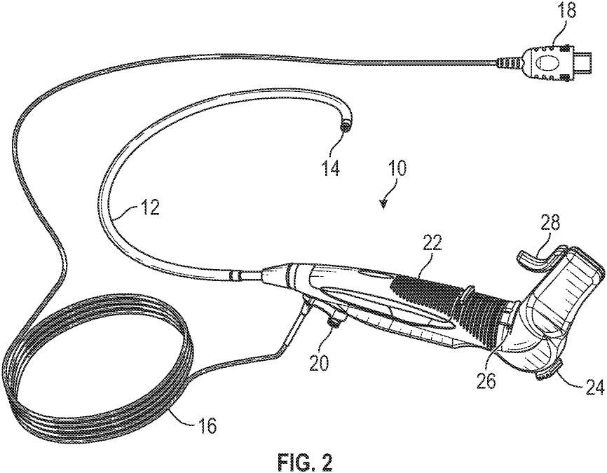

To help illustrate the techniques of this disclosure, FIG. 2 is a schematic elevational view of a cystoscope 10 having an elongated shaft 12 that defines a working channel (not shown in this view). At the distal end of shaft 12 is an imaging sensor 14 that is coupled to a cable 16 having a suitable connector 18 for interfacing with a monitor or other type of display. Shaft 12 may have different lengths based at least on criteria such as the procedure intended. For example, shaft 12 may have a working length of between 22 cm and 60 cm, preferably between 33 cm and 40 cm, and most preferably between 37 cm and 40 cm. As warranted, the length of shaft 12 may be sufficient to allow the desired view of the patient’s anatomy. An irrigation port 20, such as a luer connector, is in fluid communication with a working channel defined by shaft 12. At the proximal end of cystoscope 10 is a handle 22 that is configured to allow the operator to manipulate cystoscope 10 as its distal end is advanced though the patient. In one aspect, this includes applying rotational torque to shaft 12 via handle 22. Further, shaft 12 may be deflectable as known in the art and described in further detail below, such that the distal end may be steered to help guide the advancement through the patient. As such, handle 22 may include a steering control 24 that is coupled to one or more steering wires (not shown in this view) so that the distal end of shaft 12 is selectively deflectable to adjust the angle of imaging sensor 14 to obtain a desired view or to facilitate navigation through the patient’s anatomy. Likewise, handle 22 may also have a steering lock 26 to hold the distal end of shaft 12 in a desired degree of deflection. Still further, handle 22 features an actuator 28 that controls a movable element (not shown in this view) that engages a cartridge tool when the cartridge tool is docked in a repository of handle 22 (also not shown in this view) as detailed in the discussion below.

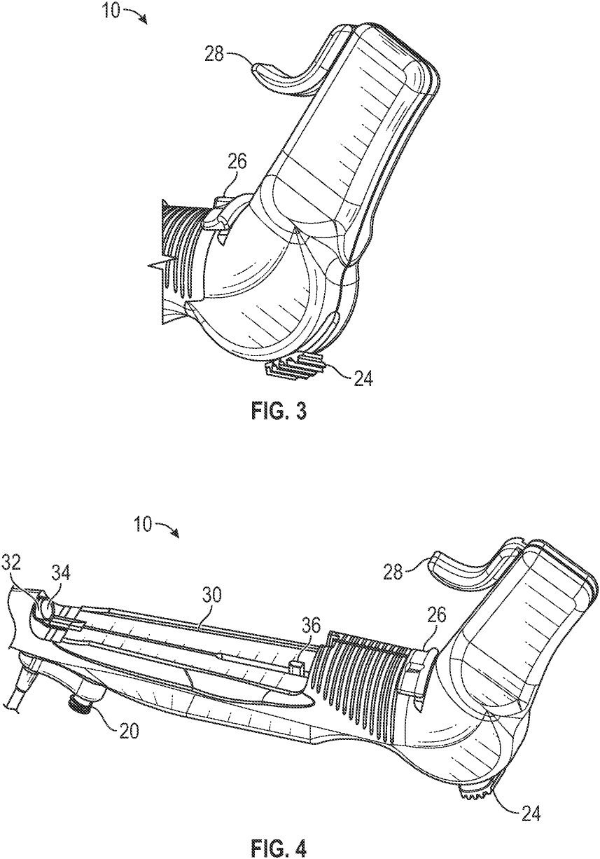

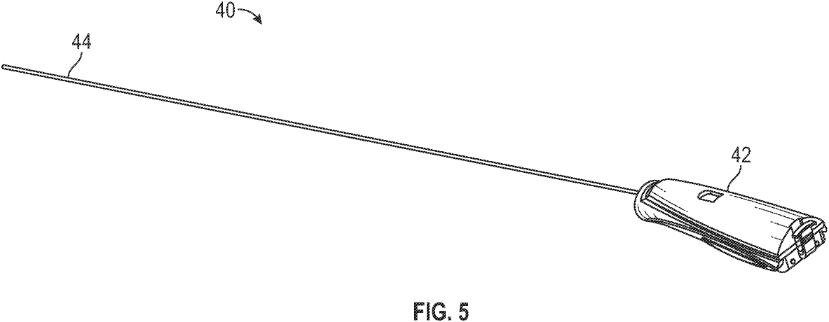

Further details of a proximal portion of handle 22 are shown in the detail view of FIG. 3 which shows the actuators, including steering control 24, steering lock 26 and cartridge tool actuator 28. Likewise, FIG. 4 is another detail view showing repository 30 that is configured as a recess or cavity defined by handle 22. Accordingly, at least a portion of the cartridge tool’s proximal end is within the profile of handle 22 when the tool is docked to help minimize bulk of the assembled system. Within repository 30 is an access 32 configured to allow a distal portion of a cartridge tool to be extended into the working channel of shaft 12 when the cartridge tool is docked in repository 30. Since irrigation port 20 is in fluid communication with the working channel of shaft 12 as noted above, access 32 may have a seal 34 to restrict the egress of irrigation fluid, particularly when a cartridge tool is docked and extends through access 32. As shown, movable element 36 is disposed within repository 30 so that it can engage a cartridge tool when docked, which is coupled to cartridge tool control actuator 28.

As will be appreciated, the techniques of this disclosure represent multiple benefits. By providing cartridge tool actuator 28 on handle 22, a single operator may simultaneously implement the functionality of the cartridge tool while also manipulating and guiding cystoscope 10, including by operating steering control 24 and/or steering lock 26. By integrating the necessary controls for both cystoscope 10 and the cartridge tool into handle 22, bulk is reduced while allowing usage by a single operator. Notably, cartridge tool actuator 28 may be used to implement the functionality of any type of cartridge tool that is docked. For example, a typical procedure may include the operator using a thumb to engage steering control 24 and an index finger to engage cartridge tool actuator 28, requiring only one hand to operate both cystoscope 10 and the cartridge tool if desired.

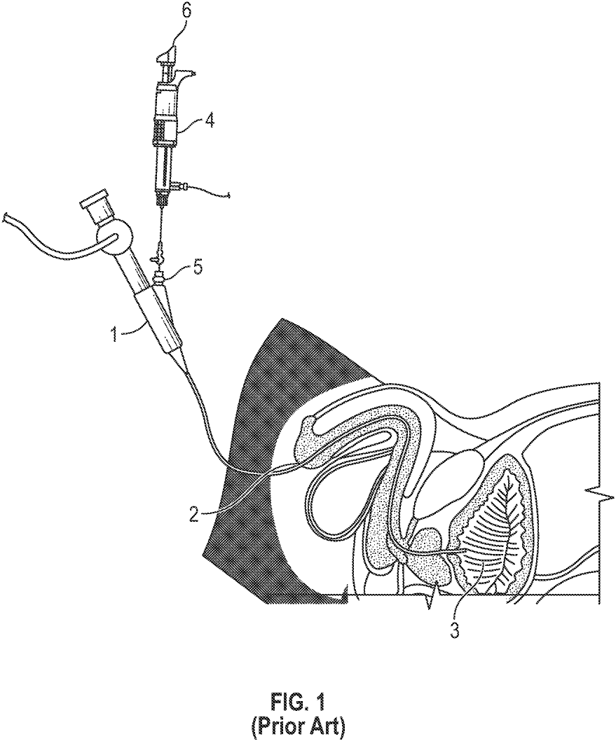

Further, irrigation port 20 represents a dedicated pathway to the working channel of shaft 12 so that irrigation needs only to be connected once at the beginning of the procedure. In contrast, conventional cystoscope designs such as those shown in FIG. 1 generally involve the separate tool employing the irrigation port, requiring reconfiguration of the irrigation pathway when the tool is being used. Further, since irrigation port 20 only connects to a suitable supply of irrigation fluid, a known and reliable connection such as a luer connector may be used so that a dedicated interface is made using seal 34 with the cartridge tool. The connection to irrigation port 20 need only be made once even if multiple cartridge tools are exchanged. Moreover, the techniques of this disclosure include employing a single working channel to supply irrigation fluid as well as to provide access for the cartridge tool so that the diameter of the working channel can be optimized for maximum irrigation flow.

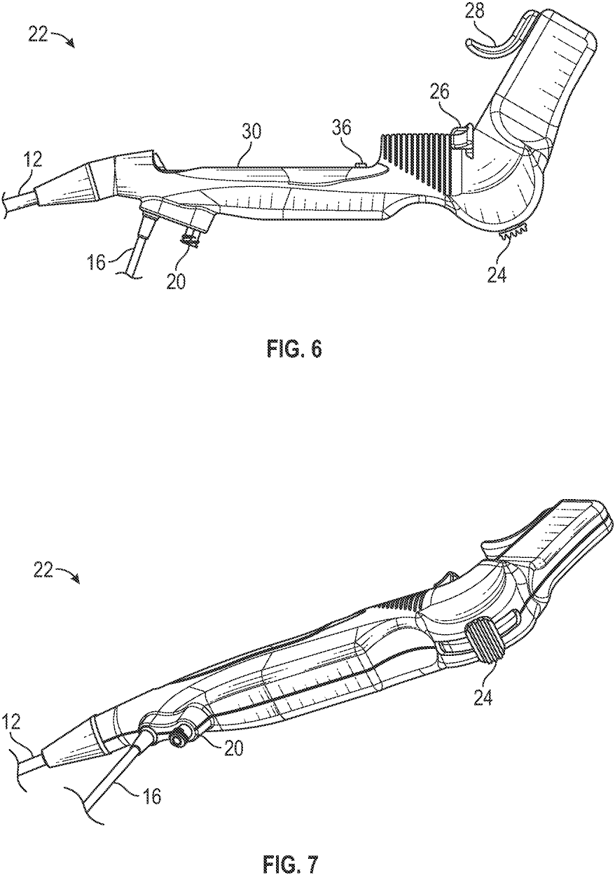

Turning now to FIG. 5, an elevational view of an exemplary cartridge tool 40 is shown. Generally, cartridge tool 40 includes a body 42 that is configured to mate with repository 30 of handle 22, such as through a snap fit connection or other suitable attachment, so that cartridge tool 40 interconnects with cystoscope 10 to allow their movement to be synchronized. However, it should also be appreciated that cystoscope 10 may already be positioned within a patient’s anatomy when cartridge tool 40 is inserted through access 32 and docked with handle 22. For example, a given procedure may involve use of multiple cartridge tools that have different functionalities or different cartridge tools may be selected to deliver different sized devices. As such, cystoscope 10 may be maintained at a desired position within the patient while different cartridge tools 40 are exchanged. Cartridge tool 40 includes an elongated distal portion 44 that is configured to extend within the working channel of shaft 12. As discussed above, cartridge tool actuator 28 is coupled to movable element 36 that engages a complimentary movable element within body 42 to implement the functionality of cartridge tool 40, which as discussed above may include deploying or retrieving a prostatic urethra implant. Thus, each of the functionalities implemented by different embodiments of cartridge tool 40 may nevertheless share the same ability to dock within repository 30 and share the same interface with cartridge tool actuator 28 and movable element 36. As will be appreciated, these characteristics reduce any learning curve for operation of cystoscope 10 since the same controls, including cartridge tool actuator 28, are used regardless of the type of cartridge tool employed. In one aspect, multiple cartridge tools 40 could be used during one procedure by re-deploying cartridge tool actuator 28 when each tool is swapped.

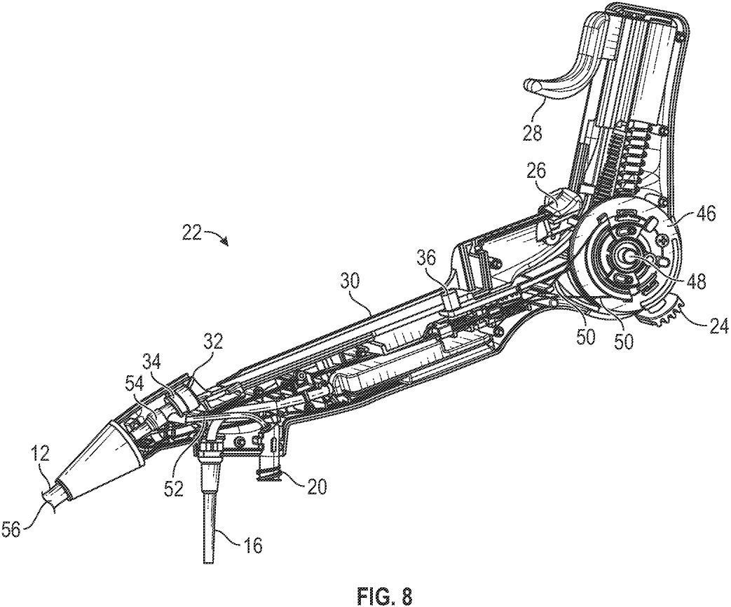

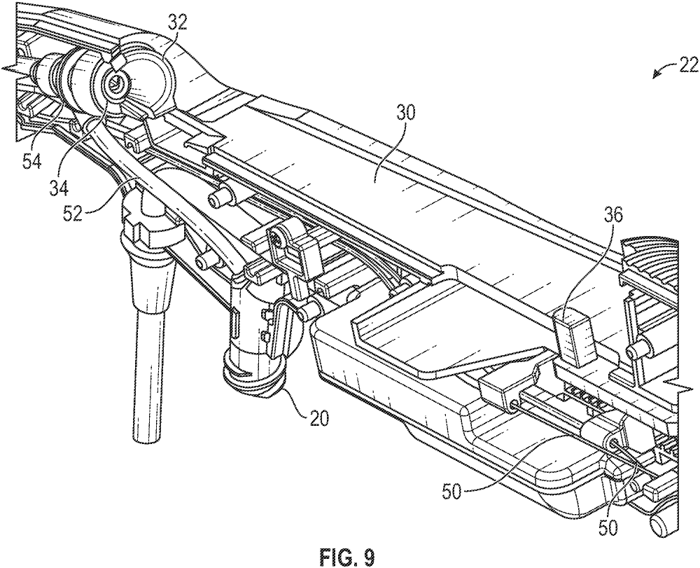

Additional views of handle 22 are shown in FIGS. 6 and 7. In the side view of FIG. 6, handle 22 is shown with repository 30 opening facing upwards and movable element 36 disposed within. Towards the distal portion of handle 22 are irrigation port 20 and cable 16 for imaging sensor 14. This view also depicts cartridge tool actuator 28 as well as steering control 24 and steering lock 26. In the bottom three quarters view of FIG. 7, irrigation port 20 and cable 16 can again be seen towards the distal portion of handle 22. Steering control 24 is shown in this embodiment to be configured as a lever positioned in the middle of its range of travel. Moving steering control 24 from this middle position causes deflection of the distal end of shaft 12 as noted above. Additional details are shown in the cut away view of FIG. 8. In particular, cartridge tool actuator 28 causes a hub 46 to rotate about an axle 48 so that tension can be selectively applied to either of steering wires 50 depending on which direction hub 46 is rotated.

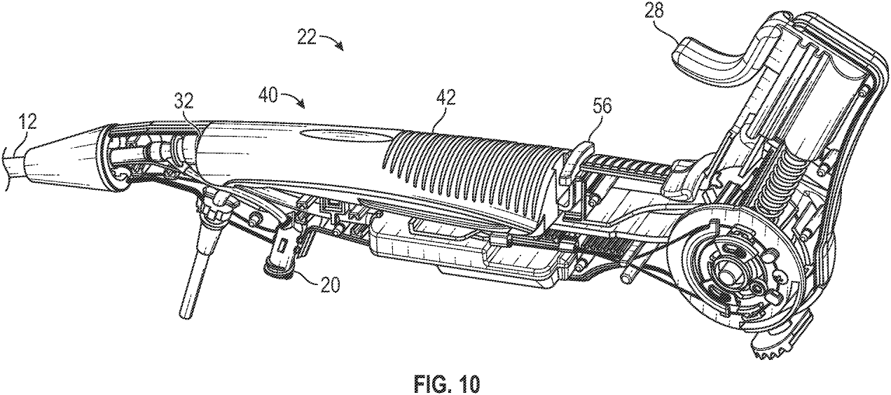

Also shown in FIG. 8 is a tube 52 coupling irrigation port 20 with a valve 54 at the proximal end of a working channel 56 that is defined by shaft 12 as noted above. At the distal end of repository 30 is access 32 and seal 34 through which distal elongated portion 44 of cartridge tool 40 extends into working channel 56 when the tool is docked. Seal 34 is configured to restrict the egress of irrigation fluid from access 32 when cartridge tool 40 is docked. Further details are depicted in the partial, cut away three quarter top view shown in FIG. 9, including the position of seal 34 within access 32. Also shown is the coupling of irrigation port through tube 52 and valve 54.

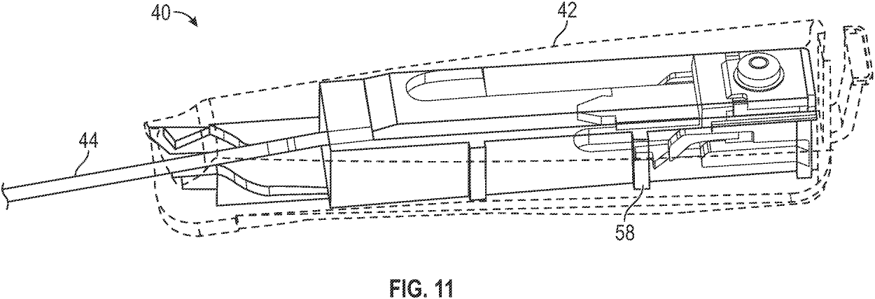

Next, FIG. 10 is another cut away view of handle 22 that shows cartridge tool 40 docked within repository 30. Elongated distal portion 44 (not shown in this view) has been introduced through access 32 and body 42 has been secured in place via releasable attachment 56. At a minimum, cartridge tool actuator 28 is used to activate movable element 36 which engages a complimentary element within cartridge tool 40 to affect its functionality. For example, FIG. 11 depicts internal components of cartridge tool 40, with body 42 shown in phantom (dashed lines). A movable element 58 within body 42 is configured to be engaged by movable element 36 of handle 22. Correspondingly, when cartridge tool actuator 28 is operated, movable element 36 is translated distally, which due to engagement, also translates movable element 58 distally. This movement causes cartridge tool 40 to implement its functionality, such as by deploying an expandable implant to restore patency to the prostatic urethra, although other cartridge tools may be configured to achieve different functionality. Depending upon the embodiment, cartridge tool 40 may have functionality involving additional controls, which may be deployed on body 42 so that the operator can manipulate them when cartridge tool 40 is docked to handle 22.

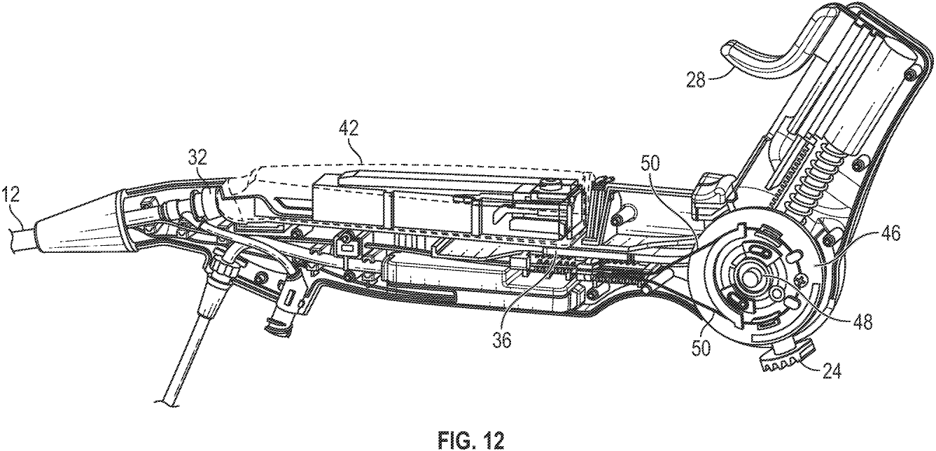

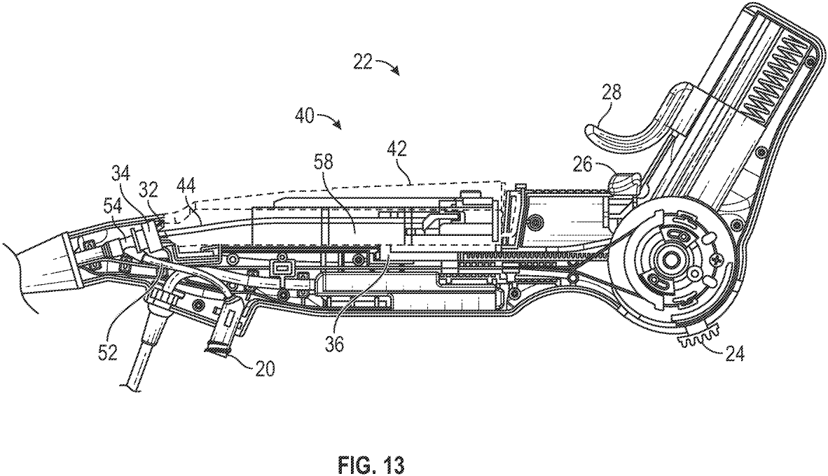

A schematic depiction of usage of cystoscope 10 and cartridge tool 40 according to techniques of this disclosure is indicated by FIGS. 12 and 13, with FIG. 12 corresponding to cartridge tool actuator 28 in an initial, undeployed position in which movable element 36 is in its most proximal position and FIG. 13 corresponding to cartridge tool actuator 28 in its fully deployed position in which movable element 36 is in its most distal position 40. In this embodiment, cartridge tool actuator 28 is configured as a plunger so that when it is fully depressed, movable element 36 travels from the proximal position shown in FIG. 12 to the distal position shown in FIG. 13. Since movable element 36 of handle 22 engages the complimentary movable element 58 in cartridge tool 40, an equivalent distal motion is produced. Cartridge tool 40 is configured to convert this translational movement into the desired functionality, such as by deploying an expandable implant. Different linkages within cartridge tool 40 may be used based upon the desired functionality, such as by converting the relative distal motion of movable element 36 to a proximal movement if warranted. Further, cartridge tool actuator 28 may employ gearing or other suitable coupling to change the ratio of travel between cartridge tool actuator 28 and movable element depending upon the intended usage.

The exemplary embodiments disclosed above are merely intended to illustrate the various utilities of this disclosure. It is understood that numerous modifications, variations and combinations of functional elements and features of the present disclosure are possible in light of the above teachings and, therefore, within the scope of the appended claims, the present disclosure may be practiced otherwise than as particularly disclosed, and the principles of this disclosure can be extended easily with appropriate modifications to other applications.

All patents and publications are herein incorporated for reference to the same extent as if each individual publication was specifically and individually indicated to be incorporated by reference. It should be understood that although the present disclosure has been specifically disclosed by preferred embodiments and optional features, modification and variation of the concepts herein disclosed may be resorted by those skilled in the art, and that such modifications and variations are considered to be within the scope of this disclosure.

What is claimed is:

1. A cystoscope comprising:
a handle;
an elongated shaft that defines at least one working channel;
a repository configured to receive a cartridge tool and provide access to the working channel; and
a first control for actuating a movable element of the repository, wherein the movable element is configured to engage a cartridge tool docked within the repository and thereby implement a functionality of the cartridge tool when the first control is actuated.

2. The cystoscope of claim 1, further comprising an irrigation port in fluid communication with the working channel, wherein the irrigation port is separate from repository.

3. The cystoscope of claim 2, wherein the repository further comprises a seal configured to restrict egress of irrigation fluid from the working channel when a cartridge tool is docked within the repository.

4. The cystoscope of claim 1, wherein the handle further comprises a steering control configured to cause selective deflection of a distal end of the elongated shaft.

5. The cystoscope of claim 4, wherein the handle further comprises a steering lock configured to maintain the distal end of the elongated shaft at a desired deflection.

6. The cystoscope of claim 1, wherein the repository comprises a recess such that at least a portion of a docked cartridge tool is within a profile of the handle.

7. The cystoscope of claim 1, further comprising at least one cartridge tool, wherein the cartridge tool has an elongated distal portion configured to extend into the working channel and has a body that releasably attaches to the repository.

8. The cystoscope of claim 7, wherein the cartridge tool has a movable element that is engaged by the movable element of the handle when the cartridge tool is docked within the repository such that manipulation of the first control implements a functionality of the cartridge tool.

9. A cystoscope system comprising:
a cystoscope having a handle, an elongated shaft that defines at least one working channel, a repository configured to receive a cartridge tool and provide access to the working channel and a first control for actuating a movable element of the repository, wherein the movable element is configured to engage a cartridge tool docked within the repository and thereby implement a functionality of the cartridge tool when the first control is actuated; and
at least one cartridge tool, wherein the cartridge tool has an elongated distal portion configured to extend into the working channel and has a body that releasably attaches to the repository.

10. The cystoscope system of claim 9, wherein the cartridge tool has a movable element that is engaged by the movable element of the handle when the cartridge tool is docked within the repository such that manipulation of the first control implements a functionality of the cartridge tool.

11. The cystoscope system of claim 9, wherein the cystoscope further comprises an irrigation port in fluid communication with the working channel and wherein the repository further comprises a seal configured to restrict egress of irrigation fluid from the working channel when the cartridge tool is docked within the repository.

12. The cystoscope system of claim 9, further comprising a plurality of cartridge tools.

13. The cystoscope system of claim 12, wherein at least two of the plurality of cartridge tools have different functionalities.

14. The cystoscope system of claim 12, wherein at least two of the plurality of cartridge tools have expandable implants of different sizes.

15. A method of employing a cystoscope comprising:
providing a cystoscope having a handle, an elongated shaft that defines at least one working channel, a repository configured to receive a cartridge tool and provide access to the working channel and a first control for actuating a movable element of the repository;
docking a cartridge tool with the cystoscope such that a distal elongated portion of the cartridge tool extends through the working channel and a body of the cartridge tool releasably attaches within the repository, wherein docking the cartridge tool causes the movable element of the repository to engage the cartridge; and
actuating the first control to implement a functionality of the cartridge tool.

16. The method of claim 15, further comprising supplying irrigation fluid to the working channel through an irrigation port, wherein docking the cartridge tool engages a seal that restricts egress of irrigation fluid from the working channel into the repository.

17. The method of claim 15, further comprising operating a steering control of the handle of the cystoscope to cause selective deflection of a distal end of the elongated shaft.

18. The method of claim 15, further comprising operating a steering lock of the handle of the cystoscope to maintain the distal end of the elongated shaft at a desired deflection.

19. The method of claim 15, wherein the cartridge tool has a movable element that is engaged by the movable element of the handle when the cartridge tool is docked within the repository such that manipulation of the first control implements a functionality of the cartridge tool.

20. The method of claim 15, further comprising exchanging the cartridge tool with a different cartridge tool.
